(19) United States
(12) Patent Application Publication
(10) Pub. No.: US 2025/0018629 A1
(43) Pub. Date: Jan. 16, 2025

(54) UHMWPE PASTE EXTRUDED TUBES

(71) Applicant: Zeus Company LLC, ORANGEBURG, SC (US)

(72) Inventors: Bhavya Singhi, Columbia, SC (US); Justin A. Marro, Orangeburg, SC (US); Robert L. Ballard, Lexington, SC (US); Patrick Cooper, Orangeburg, SC (US); Daniel Green, Orangeburg, SC (US); Douglas Lee Tourville, Orangeburg, SC (US); John Richard Campanelli, West Columbia, SC (US); Morgan Baltzegar, St. Matthews, SC (US)

(21) Appl. No.: 18/899,261

(22) Filed: Sep. 27, 2024

Related U.S. Application Data
(63) Continuation of application No. 18/238,034, filed on Aug. 25, 2023.
(60) Provisional application No. 63/401,208, filed on Aug. 26, 2022.

(51) Int. Cl. B29C 48/00; B29C 48/09; B29C 48/32; B29K 23/00; B29K 105/00; B29K 105/16; B29L 23/00

(52) U.S. Cl. CPC B29C 48/022; B29C 48/09; B29C 48/32; B29K 2023/0683; B29K 2105/16; B29K 2105/251; B29K 2995/0077; B29K 2995/0097; B29L 2023/22

(57) ABSTRACT

The disclosure generally relates to ultra high molecular weight poly (ethylene) (“UHMWPE”) tubes with an average wall thickness of 0.2 mm or less; a tensile stress at break greater than 40 MPa; and a storage modulus of greater than 500 MPa at 23° C. The disclosure further relates to preparing and using such tubes and to constructions (e.g., catheter constructions) and components thereof including such tubes.

### CROSS-REFERENCE TO RELATED APPLICATIONS

The present application is a continuation of U.S. patent application Ser. No. 18/238,034, filed Aug. 25, 2023; which application claims the benefit of U.S. Provisional Patent Application No. 63/401,208, filed Aug. 26, 2022, the disclosure of which are incorporated herein by reference in their entirety.

### FIELD OF THE INVENTION

The present application relates generally to the field of tubes, such as for use as thin wall catheter liners, comprising Ultra High Molecular Weight Poly (ethylene) (UHMWPE) and to methods relating to such tubes.

### BACKGROUND OF THE INVENTION

Vascular therapy uses minimally invasive, catheter-based procedures and specialized equipment and techniques. Catheters used in these procedures commonly employ a coating or liner on the inner wall to provide a lubricious inner surface. A lubricious inside diameter (ID) associated with these devices is beneficial in reducing friction against various catheter technologies such as stents, balloons, atherectomy or thrombectomy devices as they are pushed through the tight confines of the catheter lumen. If the catheter ID is not of sufficient lubricity, devices such as stents can cause the liner to collapse in an accordion-like manner as the devices are pushed through the catheter lumen. The effect of increased lubricity of the catheter ID is a reduced deployment force of catheter devices as they are passed through the lumen, increasing the likelihood of a successful procedure. The mechanical properties of a catheter liner are also critical to their success. For example, high tensile and yield strength may be required when certain devices (e.g., flow diversion tubes, embolization, aneurysm bridging, and scaffolding and thrombectomy devices) are passed through microcatheters in a compressed state. The compressed shape exerts an outward radial force, which causes friction with the ID, commonly making delivery of the device through the lumen difficult. On the other hand, high flexibility of a liner is often desirable when catheters must pass through vasculature that involves sharp twists and turns (e.g., cerebral vasculature and below-the-knee (BTK) applications).

Among the various materials that have been pursued as inner wall (base liner) materials for use, e.g., within such catheter devices is Polytetrafluoroethylene (PTFE) due to its excellent chemical resistance, high temperature resistance, biocompatibility and very low coefficient of friction/high lubricity. One major drawback of PTFE is that it is not radiation stable. Radiation sterilization (i.e., gamma rays or electron beams) is one of the most widely used and safe sterilization process for medical devices. Radiation sterilization improves the manufacturability of catheters as it can be quickly performed in the manufacturing line, while the ethylene oxide gas sterilization (ETO) procedure, typically used with PTFE-lined catheters, requires storage for up to 48 hours to allow the gas to diffuse out of the sterilized equipment. Also, ethylene oxide gas requires careful handling because of its flammability and toxicity. Strict handling requirements and a technically complex sterilization process makes ETO sterilization technique often undesirable. In recent times, medical regulatory organizations worldwide have also been encouraging the medical industry to minimize or replace the use of ETO with alternative sterilization methods.

While several other polymers can withstand gamma irradiation, none can match the lubricity or low coefficient of friction of PTFE. Ultra high molecular weight polyethylene (UHMWPE) comes close to doing so. UHMWPE is a linear polymer with a repeating unit of —CH₂—CH₂—. Medical grade UHMWPE has long chains with a molecular mass greater than 1×10⁶ g/mol and is a semi crystalline polymer. UHMWPE has very low coefficient of friction, excellent wear resistance, good toughness, high impact strength, high resistance to corrosive chemicals, excellent biocompatibility, and low cost. Furthermore, UHMWPE has low processing temperatures, so it can be easily bonded to a variety of other polymeric catheter components. UHMWPE has been used clinically in joint implants for over 40 years, particularly as an articular liner in total hip replacements and tibial insert in total knee replacements. One drawback of UHMWPE is its very high viscosity due to its extremely high molecular weight. UHMWPE does not flow like lower molecular weight polyethylene resins or traditional melt processable polymers when raised above its melting temperature. For this reason, many thermoplastic processing techniques, such as injection molding, screw extrusion or blow molding, are not practical for UHMWPE. U.S. Pat. No. 6,837,890 describes a catheter construction that uses expanded UHMWPE as an inner layer/liner, formed by compacting UHMWPE powder into a billet, deforming the billet through a die, and further orienting the extrudate through uniaxial or biaxial stretching to impart a microporous node and fibril structure. Such an expanded UHMWPE liner, due to its microporosity, has lower strength, the potential to absorb fluid, and lower abrasion resistance compared to unexpanded, non-porous UHMWPE liners. International Patent Application Publication No. WO2023/114080A1 discusses a method of making films and porous structures with UHMWPE using a tape calendar process. However, no practical processing technique is currently available to produce very thin, high-strength, non-porous UHMWPE tubing for catheter liners.

Gel spinning processes are widely used to process UHMWPE into high strength oriented polyolefin fiber which can be used to make articles such as ropes, tennis strings, fishing nets, filters, anti-ballistic shaped articles, medical textiles and high strength medical sutures. The gel spinning of UHMWPE traditionally involves organic solvents such as decalin, tetralin, toluene, lower alkanes, paraffin oil, mineral oil, paraffin wax, etc., of which decalin and paraffin oil are most widely used. Many of these solvents are often considered unsafe for close contact or not environmentally friendly. However, gel spinning of UHMWPE has been typically used for producing only fibers and films. See, gor example, International Patent Application Publication No. WO2019/143899A1 (describing a composition comprising UHMWPE and cyclic terpene/d-limonene at 0.001 to 15 wt %, used to form a polymeric gel that is spun to fibers, films, filters and membranes). The literature does not teach any method of producing thin wall tubing or liners at high polymer concentrations of UHMWPE.

It would be a great benefit to both medical and industrial applications if a radiation sterilizable polymeric material such as UHMWPE could be processed with an eco-friendly solvent into a thin wall tubing, liner or other polymeric structures with good mechanical properties.

### SUMMARY OF THE INVENTION

The present disclosure provides UHMWPE tubes produced by a paste extrusion method, with an average wall thickness less than 0.1 mm (preferably less than 0.05 mm), with high machine direction orientation of UHMWPE polymer chains (resulting in high tensile strength). Due to the thin walls of the disclosed tubes and their high tensile modulus, they can, in some embodiments, exhibit high ID lubricity and abrasion resistance. The combination of properties exhibited by the disclosed tubes, in various embodiments, can render them particularly suitable for use within catheters, including within catheters designed for flexibility, as the thin walls of the disclosed tubes provide for a significantly flexible liner that is sterilizable by irradiation such as by gamma or electron beam radiation sources, unlike PTFE liners. In some embodiments, the paste extruded tubing can further be oriented in machine and transverse direction to alter and/or enhance mechanical, thermal and barrier properties. Additionally, the polyethylene tubes of this invention can be used as liners for metallic tubes, like laser-cut hypotubes.

The disclosure includes, without limitation, the following embodiments:
Embodiment 1: An Ultra High Molecular Weight Poly (ethylene) (UHMWPE) tube comprising: a) an average wall thickness of 0.2 mm or less; and b) a tensile stress at break greater than 40 MPa; and c) a storage modulus of greater than 500 MPa at 23° C.
Embodiment 2: The UHMWPE tube of Embodiment 1, wherein the UHMWPE tube is prepared via extrusion of a billet comprising lubricant and UHMWPE resin through an annular die.
Embodiment 3: The UHMWPE tube of Embodiment 2, wherein the lubricant is selected from the group consisting of d-limonene, naphtha, Isopar G, Isopar M, or any combination thereof.
Embodiment 4: The UHMWPE tube of any of Embodiments 1-3, wherein the UHMWPE tube is prepared via extrusion over a metallic or non-metallic wire or mandrel.
Embodiment 5: The UHMWPE tube of Embodiment 4, wherein the metallic or non-metallic wire or mandrel and UHMWPE tube are both substantially cylindrical in shape.
Embodiment 6: The UHMWPE tube of any of Embodiments 1-5, wherein the average wall thickness is 0.1 mm or less.
Embodiment 7: The UHMWPE tube of any of Embodiments 1-5, wherein the average wall thickness of the tube is 0.005 mm to 0.1 mm.
Embodiment 8: The UHMWPE tube of any of Embodiments 1-7, wherein the tube exhibits a change in the storage modulus between 23° C. and 40° C. of 70 MPa/°° C. or less.
Embodiment 9: The UHMWPE tube of any of Embodiments 1-8, comprising an inner surface with a coefficient of friction against stainless steel of less than 0.2.
Embodiment 10: The UHMWPE tube of Embodiment 9, wherein a difference in the coefficient of friction between 23° C. and 40° C. is ≤0.1.
Embodiment 11: The UHMWPE tube of any of Embodiments 1-10, comprising an inner surface with a coefficient of friction against stainless steel in saline of less than 0.1.
Embodiment 12: The UHMWPE tube of Embodiment 11, wherein a difference in the coefficient of friction in saline between 23° C. and 40° C. is ≤0.1.
Embodiment 13: The UHMWPE tube of any of Embodiments 1-12, consisting essentially of UHMWPE.
Embodiment 14: The UHMWPE tube of any of Embodiments 1-12, comprising UHMWPE and a particulate filler, wherein the particulate filler is present in a concentration of less than 50% by weight, based on a weight of the UHMWPE tube.
Embodiment 15: The UHMWPE tube of any of Embodiments 1-12, comprising UHMWPE and a particulate filler, wherein the particulate filler is present in a concentration of less than 20% by weight, based on a weight of the UHMWPE tube.
Embodiment 16: The UHMWPE tube of Embodiment 14 or 15, wherein the particulate filler is a filler to impart radiopacity, strength, or hydrophilicity.
Embodiment 17: The UHMWPE tube of any of Embodiments 1-12 or 14-16, further comprising one or more additives selected from the group consisting of antioxidants, antimicrobials, processing aids, sip aids, and colorants.
Embodiment 18: The UHMWPE tube of any of Embodiments 1-12 or 14-17, wherein the UHMWPE tube comprises one or more additional polymeric materials other than UHMWPE, wherein the one or more additional polymer materials are present in a concentration of less than 50% by weight, based on a weight of the UHMWPE tube.
Embodiment 19: The UHMWPE tube of any of Embodiments 1-12 or 14-17, wherein the UHMWPE tube comprises one or more additional polymeric materials other than UHMWPE, wherein the one or more additional polymer materials are present in a concentration of less than 20% by weight, based on a weight of the UHMWPE tube.
Embodiment 20: The UHMWPE tube of Embodiment 18 or 19, wherein the one or more additional polymer materials are selected from modified polyethylene and ethylene vinyl acetate tie resins.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present invention will become apparent from the following.

### BRIEF DESCRIPTION OF THE DRAWING

In order to provide an understanding of the embodiments of the invention, reference is made to the appended drawing, which is not necessarily drawn to scale, and in which reference numerals refer to components of exemplary embodiments of the invention. The drawing is exemplary only, and should not be construed as limiting the invention.

FIG. 1 is a general schematic of a tube of the present disclosure, with relevant parameters, and an expanded schematic of one cross-sectional end face of the tube.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

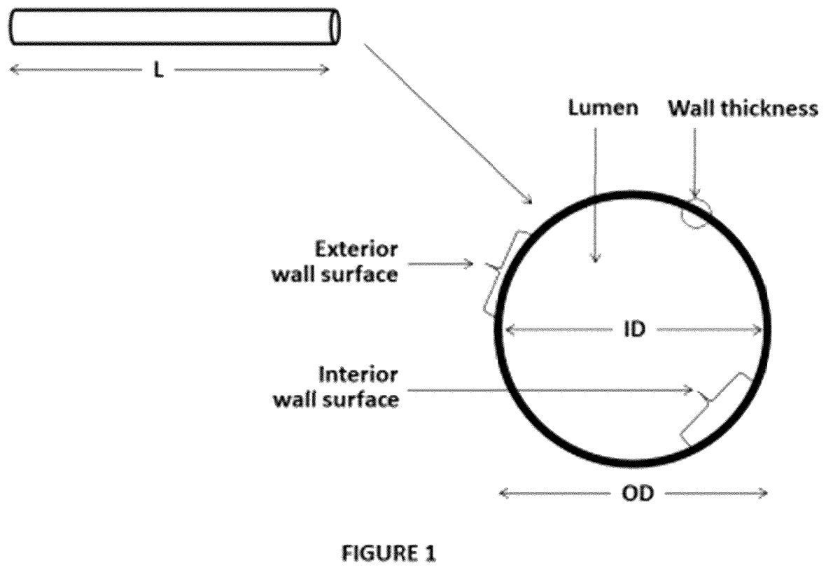

The present disclosure provides UHMWPE tubes produced by a paste extrusion method, with certain physical properties as outlined more fully herein below. A general schematic of an exemplary tube as provided is shown in FIG. 1. The tube is generally cylindrical in shape. “L” indicates the length of the tube as produced, which can be processed, e.g., cut, to provide tubes of desired length “l” (not shown). The expanded region at the right of FIG. 1 is a cross-sectional view of the interior of the tube. As shown, the “lumen” is an interior region of the tube, i.e., an open channel/cavity (through which, e.g., a catheter device may be passed when the tube is incorporated as a portion of a medical device). The inner diameter of the tube, shown as “ID” is the average distance from a point on the interior wall of the tube to the opposite/farthest point on the interior wall of the tube. The outer diameter of the tube, shown as “OD” is the average distance from a point on the outer wall of the tube through the lumen of the tube to the opposite/farthest point on the outer wall of the tube. As such, half of the value provided by subtracting the ID value from the OD value provides the average wall thickness of the tube. A representative “wall thickness.” “interior wall surface,” and “exterior wall surface” of the tube are also shown in FIG. 1.

In certain embodiments, the present disclosure provides UHMWPE tubes with thin walls (i.e., where the “wall thickness” shown in FIG. 1 is relatively small). For example, the average thickness of the walls in some embodiments is less than about than about 0.2 mm or less than 0.100 mm, preferably less than about 0.075 mm and, more preferably less than 0.050 mm. For example, the average thickness can be about 0.005 to about 0.2 mm, about 0.005 mm to about 0.1 mm, about 0.005 mm to about 0.075 mm, or about 0.005 mm to about 0.05 mm. In preferred embodiments, the walls of the disclosed tubes are substantially uniform in thickness along the length of the tube and/or around the circumference of the tube.

As noted, the length L is not particularly limited, and a tube as schematically shown in FIG. 1 can optionally be processed, e.g., cut into multiple tubes of any desired lengths l. In some embodiments, the length l of a tube provided herein is a length suitable for use in a catheter application, e.g., as a liner. For example, in some embodiments, the length l is about 150 mm to about 2000 mm. Similarly, the ID (which determines the diameter of the lumen) can vary and, in some embodiments, is of a size suitable for use in catheter applications, e.g., as a liner, such as, e.g., about 1 mm to about 11 mm.

The tubes provided herein generally comprise UHMWPE. Various UHMWPE resins are commercially available and can be used in certain embodiments within the UHMWPE tubes provided herein. In some embodiments, the UHMWPE is the only polymer within the disclosed tubes. In other embodiments, one or more other polymers can be contained within the disclosed tubes, e.g., in amounts of less than 50% by weight, less than 40% by weight, less than 30% by weight, less than 20% by weight, less than 10% by weight, less than 5% by weight, less than 1% by weight, less than 0.5% by weight, less than 0.1% by weight, less than 0.05% by weight, or less than about 0.01% by weight, based on the total weight of the tube (e.g., about 0.01% to about 50%, about 0.01% to about 30%, about 0.01% by weight to about 10%, about 0.01% by weight to about 1% by weight, or about 20% by weight to about 50% by weight. about 30% by weight to about 50% by weight, or about 40% by weight to about 50% by weight).

In some embodiments, the optional one or more additional polymers can be tie resins. In some embodiments, the optional one or more additional polymers can be, e.g., modified polyethylene and/or ethylene vinyl acetate (EVA) tie resins. In some embodiments, the optional one or more additional polymers can be, e.g., grafted polyethylene, such as maleic-anhydride grafted HDPE. Where one or more additional polymers are incorporated within the disclosed tube, it is typically in the form of an intimate mixture/blend with the UHMWPE such that the composition of the material of the tube is substantially uniform throughout. In some embodiments, a second (or further) polymer can be incorporated to modify the physical properties of the tube, e.g., mechanical properties, thermal and barrier properties, crystallinity, and/or coefficient of friction.

Advantageously in various embodiments, the tubes consist essentially of UHMWPE (alone or in combination with one or more additional polymers, as referenced above), i.e., no significant amount of additional components (e.g., fillers) are contained within the tubes In other embodiments, the disclosed tubes can comprise one or more additives or fillers, e.g., particulate fillers, in concentrations up to about 50% by weight based on the total weight of the tube, e.g., about 0.01% to about 50%, such as about 0.01% to about 10%, about 0.01% to about 10%, about 0.01% to about 20%, about 10% to about 50%, about 20% to about 50%, about 20% to about 30%, or about 30% to about 50% by weight based on the total weight of the tube. Suitable particulate fillers can vary and may include, for example, fillers designed to impart specific properties such as radiopacity, strength, and/or hydrophilicity. In some embodiments, one or more additives such as antioxidants, antimicrobials, processing aids, slip aids, and/or colorants is contained within the disclosed tubes. In some embodiments, the fillers can be thermal, oxidative, or light stabilizers.

In some embodiments, some amount of lubricant or other processing aid (such as lower molecular weight polyethylenes or waxes such as polyethylene waxes, erucamides, oleamides or stearates) can be contained within the tubes although in preferred embodiments, the tubes comprise only trace amounts or no detectable amount of lubricant or other processing aid.

In some embodiments, the UHMWPE tubes have high lubricity and abrasion resistance. Such features can be characterized in varying ways. Abrasion is generally understood to be the wearing down of a material due to friction. The friction is caused by rubbing or scraping the initial material. Abrasion resistance is a material property that prevents wear when friction is applied to the surface. Abrasion resistance is understood to be material-dependent as well as processing-dependent. There are a variety of methods used to quantify abrasion and wear depending on the sample geometry and the application. In certain embodiments, the material or specimen to be tested is positioned relative to another material such that two surfaces are contacting. The other material can be chosen based on required test conditions. Some common materials include polished metal surfaces, metal pins, sandpaper, or the same material as the test sample. Then, one or both materials are moved such that friction is caused between the contacting surfaces. Depending on the test methodology, the end of the test can be defined after a certain amount of time, a defined number of times the surfaces are rubbed, or until a specified failure mode is reached. Some failure modes include reaching: a defined mass loss, a defined reduction of material thickness relative to the initial material thickness, tensile property loss, or insulative property loss. Regardless of the test methodology selected, the relative abrasion resistance of two or more materials can be determined by subjecting the samples to the same test conditions and comparing the impact of the test conditions on the materials tested. Some methods to detect abrasion of the samples are weight loss, visual check, and microscope images. Various methods to evaluate abrasion resistance that can be used to define tubes of the present disclosure include, but are not limited to, those disclosed which are incorporated herein by reference (including, e.g., pin abrasion testing (e.g., using ASTM G132), rubber wheel abrasion testing (e.g., using ASTM G65), taber abrasion testing (e.g., using ASTM D1044 and ASTM D4060), blade-on-block wear testing, medical device wear testing, and pin-on-disk wear testing (e.g., using ASTM G99, ASTM G133, and ASTM F732)). One further exemplary method is EN 3475 Method 511, which generally comprises placing the material in a fixture so that it is contacting another sample of the same type; the ends are fixed in place and the sample is vibrated/rubbed against itself.

Suitable abrasion resistance/lubricity values for the disclosed tubes can vary, but advantageously, such values are rather high. For example, in some embodiments, at least the interior wall surface of the disclosed tubes can be described as being “abrasion resistant” and/or “lubricious” and in some embodiments, both the interior wall surface and the exterior wall surface can be similarly described. For example, in some embodiments, the interior and/or exterior wall of the tube is a lubricious surface with a coefficient of friction of less than about 0.2 or less than about 0.1 (e.g., about 0.05 to about 0.2 or about 0.05 to about 0.1). In some embodiments, the interior and/or exterior wall of the tube is a lubricious surface with a coefficient of friction in saline of less than about 0.2 or less than about 0.1 (e.g., about 0.02 to about 0.2 or about 0.02 to about 0.1). In some embodiments, the lubricious surface(s) of the disclosed tubes exhibit a difference in coefficient of friction between 23° C. and 40° C. of less than or equal to about 0.1 (e.g., about 0.01 to about 0.1). In some embodiments, the lubricious surface(s) of the disclosed tubes exhibit a difference in coefficient of friction in saline between 23° C. and 40° C. of less than or equal to about 0.1 (e.g., about 0.01 to about 0.1). Such values as given above can be, e.g., coefficients of friction against stainless steel.

In some embodiments, the disclosed tubes exhibit physical properties that render them suitable for a wide range of applications. In some embodiments, the disclosed tubes exhibit high tensile stress at break values, e.g., greater than 40 MPa. For example, in some embodiments, the tubes exhibit tensile stress at break values of about 40 MPa to 400 MPa, e.g., 40 MPa to 200 MPa. In some embodiments, the disclosed tubes exhibit high storage modulus values. For example, in some embodiments, the disclosed tubes can exhibit a storage modulus of greater than 500 MPa at 23° C., e.g., in some embodiments, greater than 750 MPa at 23° C., greater than 1,000 MPa at 23° C. 1,200 MPa at 23° C., greater than 1,500 MPa at 23° C., greater than 2,000 MPa at 23° C., greater than 2,200 MPa at 23° C., or greater than 2,500 MPa at 23° C. e.g., about 500 MPa to about 5,000 MPa, about 500 MPa to about 2,500 MPa, or about 1,000 MPa to about 5,000 MPa at 23° C. In some embodiments, the change in storage modulus is rather low, e.g., such that the tube exhibits a change in the storage modulus between 23° C. and 40° C. of 500 MPa/° C. or less, 200 MPa/° C. or less, 100 MPa/° C. or less, or 70 MPa/° C. or less, e.g., about 10 MPa/° C. to 500 MPa/° C. or about 10 MPa/° C. to 70 MPa/° C.

In some embodiments, the disclosed tubes can be referred to as “paste extruded” tubes. The method of preparing these tubes (as referenced herein below) results in certain physical properties that distinguish such tubes, e.g., from free-extruded tubes. For example, in some embodiments, tubes uniquely prepared via this method (e.g., paste extruding UHMWPE over a substrate/core and subsequently sintering and removing the resulting UHMWPE tube from the substrate/core) exhibit low machine-direction orientation of UHMWPE polymer chains, e.g., much lower than that exhibited by free extruded tubes. Further, the tubes uniquely can exhibit particularly beneficial properties (e.g., which may result, at least in part, from the method of production), including, e.g., exceptional strength and flexibility features even at very low wall thicknesses.

The produced UHMWPE tube can be described, in some embodiments, as “unstretched” and/or “undrawn,” i.e., it has not been stretched following production to impart molecular orientation. In some embodiments, the produced UHMWPE tube can be described as “stretched” and/or “drawn” following production, and thus can exhibit machine-direction molecular orientation and, correspondingly, an increased tensile strength and reduced wall thickness (relative to the corresponding as-produced tube). In some embodiments, the produced UHMWPE tube can be described as “stretched” and/or “drawn” in machine and transverse directions.

It is noted that the properties of a given tube may, in some embodiments, vary somewhat from the disclosed properties where fillers and/or other polymers are contained therein. For example, in some embodiments, tubes including one or more tie resins as referenced above can enhance or impart one or more specific properties such as lubrication, toughness, or adhesion. In some embodiments, tubes including one or more added fillers and/or polymers other than UHMWPE may have different properties, such as mechanical, thermal and barrier properties, crystallinity and coefficient of friction, etc. Further, the inclusion of e.g., an antioxidant or antimicrobial will lend corresponding features to the tube and colorant will lend the relevant color to the tube.

The disclosure further provides a method for producing UHMWPE profiles, e.g., monofilaments, multifilaments, ribbons, and tubes exhibiting the physical properties described herein above. The method generally comprises employing a paste extrusion process, wherein UHMWPE is extruded freely or over a metallic or non-metallic substrate, e.g., including, but not limited to, a wire or PTFE core/mandrel. Paste extrusion methods for UHMWPE may involve several steps, including: (1) paste preparation or resin mixing with lubricants; (2) preform preparation; (3) extrusion; (4) lubricant evaporation; and (5) curing/annealing/sintering, as referenced in further detail below. It is to be understood that, for some paste extrusion methods, the preparation of a preform prior to extrusion is not required.

Fine powder UHMWPE resins that are suitable for the extrusion process disclosed herein arc typically homopolymer usually having a molecular weight greater than 1×106 g/mol (usually calculated from IV/intrinsic viscosity measurements). Exemplary resins suitable for this purpose include, but are not limited to, Celanese's [GUR®2024, GUR®2122, GUR®2122-5, GUR® 2126, GUR® 4012, GUR®4012 F, GUR® 4020-3, GUR® 4022, GUR® 4022-6, GUR® 4032, GUR® 4050-3, GUR® 4056-3, GUR® 4112, GUR® 4113, GUR® 4120, GUR® 4122, GUR® 4122-5, GUR®4130, GUR®4150, GUR®4150-3, GUR®4152, GUR®4170, GUR®4523, GUR®4550, GUR®5113, GUR®5129, GUR®5523, GUR®X161, GUR® X 195, GUR®X204, GUR®X 214, GUR®X217], Mitsui's [ Mipelon PM200, XM220, XM221U, XM330]. Hi-Zex Million 030S, 145M, 240S, 320MU, 630M, Braskem's UTEC3040, UTEC3041, UTEC4040, UTEC4041, UTEC5540, UTEC5541, UTEC6540, UTEC6540G, UTEC6541, Rochling's Polystone, LyondellBasell's Lupolen UHM 5000, and Asahi Kasei's Sunfine UH. Copolymers of ethylene or other polyethylene resins, such as irradiated or chemically modified polyethylene resins, may also be used in the extrusion process.

It is understood in the following discussion that the term “lubricant” can apply to any compound that at least partially wets the UHMWPE resin. Wettability/surface tension and viscosity are two properties of lubricants that can have a significant impact on the pressure of UHMWPE paste extrusion and thus can be modified accordingly. A lubricant that wets UHMWPE more helps reduce the extruder pressure. Similarly, a lubricant with low viscosity can help to reduce the extruder pressure.

The paste preparation step involves mixing of the UHMWPE fine powders with an appropriate lubricant (or a combination of lubricants). Non-limiting examples of suitable lubricants include xylene, cyclohexane, benzene, toluene, carbon tetrachloride, tetrahydrofuran, chloroform, dodecane, naphthalene, naphtha, Isopar G, Isopar M, p-xylene, 1,2,4-trichlorobenzene, kerosene, camphene, paraffin oil, decalin, polybutene, sunflower oil, palm oil, orange oil (terpene), other oleophilic hydrocarbons, and combinations thereof. An environmentally friendly solvent such as D-limonene [CAS number 5989-27-5] can also be used for the above paste preparation step. D-limonene is a cyclic terpene found in citrus extract/oil and also knows as limonene or l-limonene or dl-limonene or (+)-limonene or (+)-dipentene or (+)-(R)-limonene or (R)-4-isopropenyl-1-methyl-1-cyclohexene.

The ratio of resin to lubricant can vary from 1:1 to 15:1 (wt. in grams/vol. in mL). To form a proper paste, the polymer can be mixed with the lubricant through mechanical agitation with or without applying heat (as high as 100° C.). The paste may go through an optional filtration step to remove large agglomerates. Additionally, the paste may optionally be aged over an extended period of time, with or without heat (as high as 100° C.) to enhance wettability of the resin with the lubricant. Before the extrusion step, it is generally important to remove at least some air (advantageously, as much air as possible) from the UHMWPE paste in order to prevent defects in the extrudate. Unlike PTFE paste extrusion the preforming step in UHMWPE may not always be necessary. Instead, the paste could be loaded into the machine barrel in a single step and pressed to eliminate entrapped air. If the preforming step is used, then the mixture is typically pressed into solid or hollow shapes (e.g., cylindrical or annular profiles), which are called preforms or billets. These preforms are typically quite weak and can easily break or deform and should be handled with care. Multi-layered preforms with separate paste formulations for each layer, such as a core-sheath structure, can be used to have different materials and/or properties on the inner layer and outer layer of an extruded tubing profile. In some embodiments, multiple preforms with different lubricants or different amounts of lubricant can be made and loaded into the extruder. In such embodiments, different sections of the preform have different lubricants or different amounts of lubricant, altering the extrusion pressure during the production process.

In certain embodiments, the cylindrical preform is inserted into the extrusion cylinder/barrel of a paste extruder and then pressed through a die with the help of a ram. The barrel and/or the die can be at ambient temperatures or heated to temperatures below the resin degradation temperature. Extrusion of tubes (with or without a core) generally requires the presence of a mandrel in the barrel, which is attached to the back part. According to the present disclosure, the metallic/non-metallic, smooth or textured core is fed through this mandrel. Such core could be at room temperature or pre-heated greater than room temperature before going into the mandrel. The material of the core employed in this paste extrusion process is not particularly limited and, in some embodiments, may be metallic or non-metallic. If core coating is not intended, free extruded tubing can still be produced if the mandrel is present as mentioned earlier.

The extruded paste material coats the core (in case of core coating) that is guided through the extruder head at the same time. If the extrusion pressure changes during processing and the coating thickness (or wall thickness of free extruded tube) deviates from the target, the machine design ensures that ram speeds could be adjusted (manually/automatically) to ensure a uniform coating over the mandrel or wall thickness of free extruded tube.

During the extrusion processing, molecular/chain orientation can be imparted into the extruded product (e.g., the final part) based on the drawdown of the material. Orientation imparted into a material is known to impact tensile properties-especially the modulus, tensile strength, and elongation. With the changes noted in modulus, Coefficient of Friction (COF) may be altered as well. Generally, increasing the drawdown increases the axial orientation of the polymer, thereby increasing the modulus & tensile strength and reducing the elongation & COF. The extrudate can optionally be processed, e.g., by cutting the long tube into shorter lengths as desired, e.g., for certain specific applications.

After the extrusion, the residual lubricant in the extruded UHMWPE tubing or profile must be removed completely by heating above a boiling point or flash point of the lubricant. This step is performed, for example, by passing the product through a devolatilization oven.

After the devolatilization step, the product is heated in a higher temperature sintering/annealing oven, which is usually set at a temperature equal or higher than the melting point of UHMWPE. Depending on the line speed and thickness of the UHMWPE, the oven is generally set at well above this melting temperature. It is important that the product within the sintering oven is completely free of lubricant/solvent. In the sintering oven, the UHMWPE particles melt and adhere to each other. As the product is cooled (e.g., upon exiting/being removed from the sintering oven), the UHMWPE goes from a molten state to a solid one. The secondary stretching/orientation (in MD/TD) could also be performed the same way as mentioned earlier. Furthermore, tie material can be coated or extruded on to the UHMWPE layer to aid in bonding with materials such as PI, PU, Nylon or PEBA.

Devolatilization, sintering/annealing, stretching/orientation operations can, in another embodiment, be performed in the same oven.

The inventors have found that, by extruding UHMWPE over a substrate/core, machine-direction orientation of UHMWPE polymer chains is reduced. This feature is in contrast to, e.g., free extruded tubes, which exhibit high machine-direction orientation. By extruding and sintering UHMWPE coatings in this way and then removing the coating, tubes exhibiting particularly beneficial combinations of properties (as outlined herein above) can be readily obtained. In particular, the inventors have unexpectedly found that tubes with very low wall thicknesses can be obtained in this manner, which exhibit the strength and flexibility features outlined above, providing a particularly beneficial means for the production of very thin-walled UHMWPE tubes, e.g., for use in catheter applications.

The produced UHMWPE tube or profile can also optionally be stretched and drawn in machine direction (with or without application of heat) to impart molecular orientation and thus increase the tensile strength and also reduce the wall thickness further. The machine direction oriented tubing can then be further oriented (with or without application of heat) by stretching, for example, 1.1 to 10 times in TD (transverse direction) mechanically or pneumatically (e.g. applying air in ID). Alternately the un-stretched tubing can be oriented simultaneously in machine and transverse direction (with or without application of heat) mechanically/pneumatically or in a combination (e.g. balloon blowing machine). The stretching process can be used to produce different structural and mechanical effects in the tubes.

In some embodiments, UHMWPE can be extruded into various profiles, such as monofilaments. tubes, and ribbons, without the use of a lubricant. One or more UHMWPE resins, along with any desirable fillers such as tie-resin, can be loaded into the extruder barrel with or without making a preform. The resin material can then be extruded by pushing through a die at high temperatures (greater than melt temperatures of the resin) and subjected to additional steps, such as annealing, stretching, orientation, etc.

It is to be understood that any of the UHMWPE tubes of the invention can contain fillers to impart specific properties such as radiopacity, strength and hydrophilicity. It is also understood that any of the UHMWPE tubes of the invention can contain one or more polymers besides UHMWPE, such as modified polyethylene (maleic anhydride grafted/copolymerized polyethylenes) and ethylene vinyl acetate (EVA) tie-resins, to impart specific properties such as lubrication, toughness, or adhesion. The tubes with added fillers and polymers other than UHMWPE may have different properties, such as mechanical, thermal and barrier properties, crystallinity and coefficient of friction, etc. It is to be understood that the tubes of the invention may contain additives such as antioxidants, antimicrobials, processing aids, slip aids and colorants, as well as other particulates designed to impart specific properties to the tubes.

The extruded tubes can be subjected to radiation such as e-beam or gamma processing, with a dosage between 50 kGy to 15 MGy. The radiation treatment can alter the polymeric chain structure of the tubes and affect some physical, mechanical, or thermal properties of the liners, such as lubricity, tenacity, modulus, etc. Typically, radiation treatment can impart crosslinking in polyethylene chains, which increases the modulus and tensile strength. Alternately, irradiated UHMWPE resin can be added as a filler to the UHMWPE paste/preform to impart and/or enhance specific functionalities. The extruded tubes can also be subjected to surface modification, such as plasma treatment to improve the bonding to other polymeric materials, such as catheter jackets.

In some embodiments, a tube as disclosed herein is particularly advantageous for use as a catheter liner. A conventional three-layer catheter comprises a liner as the inner-most layer, for which the tubing of the present invention is particularly useful. The liner, by itself typically has a wall thickness between about 0.025 mm and about 0.070 mm and an inside diameter between about 0.380 mm and about 4.300 mm. A braided layer envelopes the liner, and a jacket encloses the braided envelope. The braided layer can be built with wires or filaments, of metallic or non-metallic materials, such as stainless steel, liquid crystal polymer. UHMWPE, etc. As such, the disclosure provides not only tubes as described herein, but also tubes configured as liners (e.g., which may comprise only the tube or which may comprise one or more additional components in addition to the tube, e.g., a braided layer and/or additional layers associated with a catheter construction such as jackets).

For building catheters using the disclosed tubes, instruments such as the Beahm 810A vertical laminator can be used. The polyethylene tubes provided herein can be bonded with catheter jackets made with materials such as PEBA (polyether block amide), nylon, polyurethane, etc. The disclosed tubes can also be stretched to reduce the wall thickness of the tubing, without lowering their bondability and then used to build catheters. The degree of stretching of the tubing can affect some physical, mechanical or thermal properties of the liners, such as lubricity, tenacity, modulus, etc. Typically, increasing the degree of stretching increases the axial orientation of the tubes, thereby increasing the modulus and tensile strength and reducing the elongation. Built catheters and catheter components such as liners and jackets can be tested using an interventional device testing equipment such as the IDTE3000 from MSI which can measure and record device performance features such as pushability, flexibility, torqueability, etc.

The lubricants, solvents and UHMWPE resins used in the paste extrusion methods can also be used in different concentrations to produce monofilaments for 3-D printing of polymeric structures/devices/products for various applications.

### Experimental

An Instron 5965 dual column mechanical tester running Bluehill 3 v3.73.4823 operating software was used to determine the tensile properties of the UHMWPE samples. For the ribbon samples, the test was performed at a rate of 25.4 mm/min using a 1kN load cell and a 12.7 mm gage length with Type V dogbones. The tensile test for the tubing samples was conducted using 50.8 mm gage length and 50.8 mm/min test rate and a 1 kN load cell. The mechanical properties of the monofilament samples were measure using different test parameters, as noted in the specific examples. The average tensile values of the UHMWPE profiles are listed in Table 2 and Table 3.

A TA instruments Q800 DMA with the film tension fixture was used to determine the thermo-mechanical properties of the UHMWPE profiles. The main property of interest was storage modulus (E′). A temperature scan was performed from −100° C. to 130° C. with an isothermal hold for five minutes at −100° C. The sample was heated at a constant rate of 3° C./min while being displaced at a constant amplitude of 15 μm with a fixed frequency tensile oscillation of 1 Hz. The resulting DMA data was imported into TA instruments TRIOS software v4.3, and the average values of the storage modulus at 23° C. and 40° C. are listed in Table 2 and Table 3.

A TA instruments Q800 DMA with a 3-point bend fixture was used to determine the flexural properties of the UHMWPE tubing samples. The test was operated at room temperature using a sample size of 5 mm×50 mm and span length of 15 mm with a strain ramp of 1%/min up to 5% strain. The results are listed in Table 3.

A TA instruments Discovery Hybrid Rheometer (DHR-3) rheometer with the tribo-rheometer accessory was used to determine the tribological properties of the UHMWPE samples. The main property of interest during this test was the COF. The samples were prepared by attaching three tubing sections of 5 mm×16.5 mm each to the three teeth of the half-ring for use with a Ring-on-Plate tribo-rheometry fixture. The ring with mounted samples was then attached to the ring-on-plate upper-geometry holder and lowered to have the samples contact a mirror-finish stainless steel plate at the specified axial force. Tribological tests were performed at room temperature (23° C.) from sliding speeds of 750 μm/s to 7650 μm/s under an axial load of IN. Additional tribological tests were performed in a saline bath at room temperature (23° C.), from sliding speeds of 750 μm/s to 7650 μm/s under an axial load of IN. The minimum COF over the stated range in sliding speed was calculated by the TA instruments TRIOS software v4.3. Multiple specimens were tested for each sample, and the averages are listed in Table 2 and Table 3.

A combination of sufficient parameters such as COF, tensile strength, modulus and flexural stress are general important for liners for catheter applications. As a non-limiting example, for liners used in neurovascular applications, a combination of low COF, high strength, and low flexural stress is often desirable.

### EXAMPLES

Aspects of the present invention are more fully illustrated by the following examples, which are set forth to illustrate certain aspects of the present invention and are not to be construed as limiting thereof.

Multiple UHMWPE samples with different profiles were prepared using a Malvern Advanced Capillary Rheometer RH7, a vertical paste extruder, and a horizontal paste extruder according to the specific example writeups provided below. Further information about the resin (or resins) used for these examples is provided below in Table 1. The samples were subjected to mechanical and lubricity testing according to the methods described above. Any alteration in methods for sample production or testing is noted in the specific example. The unit of measurement for weight was grams and for volume was mililitres in all the examples.

### Ribbon and Monofilament Examples

### Example 1

UHMWPE PM-200 was mixed with d-Limonene lubricant in 1:1 ratio (wt/vol) in a jar. The mixture was added into the rheometer barrel and a ribbon die with length 10.8 mm and width of 1.5 mm was used for extruding flat ribbon profiles. All the heating zones in the rheometer, including the barrel and die were set to a temperature of 165° C. The extruded ribbons were heat drawn about 200% under tension in a secondary process at around 110° C.

### Example 2

UHMWPE PM-200 was mixed with 2% PTFE (wt/wt) of the polymers in a jar. d-Limonene lubricant was added to the resin mixture in 1:1 ratio (wt/vol). The mixture was added into the rheometer barrel and a ribbon die with length 10.8 mm and width of 1.5 mm was used for extruding flat ribbon profiles. All the heating zones in the rheometer, including the barrel and die were set to a temperature of 165° C. The extruded ribbons were heat drawn about 200% under tension in a secondary process at around 110° C.

### Example 3

UHMWPE PM-200 was mixed with 5% PTFE (wt/wt) of the polymers in a jar. d-Limonene lubricant was added to the resin mixture in 1:1 ratio (wt/vol). The mixture was added into the rheometer barrel and a ribbon die with length 10.8 mm and width of 1.5 mm was used for extruding flat ribbon profiles. All the heating zones in the rheometer, including the barrel and die were set to a temperature of 165° C. The extruded ribbons were heat drawn about 200% under tension in a secondary process at around 110° C.

### Example 4

UHMWPE PM-200 was mixed with d-Limonene lubricant in 1:1 ratio (wt/vol) in a jar. The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 300% under tension in a secondary process at around 110° C.

### Example 5

UHMWPE PM-200 was mixed with 2% PTFE (wt/wt) of the polymers in a jar. d-Limonene lubricant was added to the resin mixture in 1:1 ratio (wt/vol). The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 300% under tension in a secondary process at around 110° C.

### Example 6

UHMWPE PM-200 was mixed with 5% PTFE (wt/wt) of the polymers in a jar. d-Limonene lubricant was added to the resin mixture in 1:1 ratio (wt/vol). The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 300% under tension in a secondary process at around 110° C.

### Example 7

UHMWPE PM-200 was mixed with d-Limonene lubricant in 2:1 ratio (wt/vol) in a jar. The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 400% under tension in a secondary process at around 110° C.

### Example 8

UHMWPE PM-200 was mixed with 10% EVA (wt/wt) of the polymers in a jar. d-Limonene lubricant was added to the resin mixture in 2:1 ratio (wt/vol). The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 400% under tension in a secondary process at around 110° C.

### Example 9

UHMWPE PM-200 was mixed with 10% UHMWPE XM-221 U (wt/wt) of the polymers in a jar. d-Limonene lubricant was added to the resin mixture in 2:1 ratio (wt/vol). The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 400% under tension in a secondary process at around 110° C.

### Example 10

UHMWPE PM-200 was mixed with 10% irradiated (10MRad) UHMWPE PM-200 (wt/wt) of the polymers in a jar. d-Limonene lubricant was added to the resin mixture in 2:1 ratio (wt/vol). The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 400% under tension in a secondary process at around 110° C.

### Example 11

UHMWPE PM-200 was mixed with d-Limonene lubricant in 2:1 ratio (wt/vol) in a jar. The mixture was added into the rheometer barrel and a ribbon die with length 10.8 mm and width of 1.5 mm was used for extruding flat ribbon profiles. Only the die heating zone in the rheometer was turned on and set to a temperature of 165° C. The extruded ribbons were heat drawn about 400% under tension in a secondary process at around 110° C. The drawn ribbons were subjected to ebeam irradiation with a dosage of 10 MRad.

### Example 12

UHMWPE GUR 4056-3 was compressed in a preform press into a solid cylindrical billet without any lubricant. The billet was loaded into a vertical paste extruder with a ribbon die with length 12.7 mm and width of 3.17 mm was used for extruding flat ribbon profiles. Only the die in the extruder was heated to a set temperature of 260° C. The extruded ribbons were heat drawn about 200% under tension in a secondary process at around 130° C.

### Example 13

UHMWPE GUR 4056-3 was mixed with Isopar M lubricant in 4:1 ratio (wt/vol) in a jar. The mixture was compressed in a preform press into a solid cylindrical billet. The billet was loaded into a vertical paste extruder with a ribbon die with length 12.7 mm and width of 3.17 mm was used for extruding flat ribbon profiles. Only the die in the extruder was heated to a set temperature of 260° C. The extruded ribbons were heat drawn about 300% under tension in a secondary process at around 130° C.

### Example 14

UHMWPE GUR 4056-3 was mixed with Isopar G lubricant in 10:1 ratio (wt/vol) in a jar. The mixture was compressed in a preform press into a solid cylindrical billet. The billet was loaded into a vertical paste extruder with a ribbon die with length 12.7 mm and width of 3.17 mm was used for extruding flat ribbon profiles. Only the die in the extruder was heated to a set temperature of 260° C. The extruded ribbons were heat drawn about 200% under tension in a secondary process at around 130° C.

### Example 15

UHMWPE GUR 4022-6 was mixed with Isopar M lubricant in 4:1 ratio (wt/vol) in a jar. The mixture was compressed in a preform press into a solid cylindrical billet. The billet was loaded into a vertical paste extruder with a ribbon die with length 12.7 mm and width of 3.17 mm was used for extruding flat ribbon profiles. Only the die in the extruder was heated to a set temperature of 260° C. The extruded ribbons were heat drawn about 300% under tension in a secondary process at around 130° C.

### Example 16

UHMWPE PM 200 and irradiated UHMWPE PM 200 (10 MRad) resins were mixed in a 4:1ratio (wt/wt) in a jar and 5% (wt/wt) Epolene C-16P was added to the UHMWPE resin mixture. d-Limonene was added as a lubricant to the resin mixture in 3:1 ratio (wt/vol). The mixture was compressed in a preform press into a hollow cylindrical billet. The billet was loaded into a horizontal paste extruder and a ribbon die with length 19.95 mm and width of 0.09 mm was used for extruding ribbons. The die had multiple heating zones, which were heated to set temperatures between 30° C. to 160° C. The ribbons were subjected to testing as extruded without any additional draw-down process.

### Example 17

UHMWPE PM-200 was mixed with d-Limonene lubricant in 5:1 ratio (wt/vol) in a jar. The mixture was added into the rheometer barrel and a 1.5 mm die was used for extruding monofilaments. Only the die heating zone in the rheometer was turned on and set to a temperature of 165° C. The extruded monofilaments were heat drawn about 200% under tension in a secondary process at around 110° C.

### Example 18

UHMWPE PM-200 was mixed with d-Limonene lubricant in 2:1 ratio (wt/vol) in a jar. The mixture was added into the rheometer barrel and a 1.5 mm die was used for extruding monofilaments. Only the die heating zone in the rheometer was turned on and set to a temperature of 165° C. The extruded monofilaments were heat drawn about 700% under tension in a secondary process at around 110° C. The tensile test was conducted on the Instron using 152.4 mm gage length and 304.8 mm/min test rate.

### Example 19

UHMWPE PM-200 was mixed with d-Limonene lubricant in 2:1 ratio (wt/vol) in a jar. The mixture was compressed in a preform press into a solid cylindrical billet. The billet was loaded into a horizontal paste extruder with a 2.3 mm die used for extruding monofilaments. Only the die in the extruder was heated to a set temperature of 200° C. The extruded monofilaments were heat drawn about 1000% under tension in a secondary process at around 110° C. The tensile test was conducted on the Instron using 101.6 mm gage length and 203.2 mm/min test rate.

### Example 20

UHMWPE PM-200 was compressed in a preform press into a solid cylindrical billet without any lubricant. The billet was loaded into a vertical paste extruder with a 7.37 mm die used for extruding monofilaments. Only the die in the extruder was heated to a set temperature of 300° C. The extruded monofilaments were heat drawn 200% under tension in a secondary process at around 120° C.

### Example 21

UHMWPE GUR 4022-6 was compressed in a preform press into a solid cylindrical billet without any lubricant. The billet was loaded into a vertical paste extruder with a 0.635 mm die used for extruding monofilaments. Only the die in the extruder was heated to a set temperature of 260° C. The extruded ribbons were heat drawn 200% under tension in a secondary process at around 130° C. The tensile test was conducted on the Instron using 25.4 mm gage length and 12.7 mm/min test rate.

### Tubing Examples

### Example 22

UHMWPE PM-200 was mixed with d-Limonene lubricant in 2:1 ratio (wt/vol) in a jar. The mixture was added into the rheometer barrel and a 6.35 mm spider die with a 4.83 mm mandrel was used for extruding large size tubing. Only the die heating zone in the rheometer was turned on and set to a temperature of 165° C. The extruded tubes were heat drawn about 400% under tension in a secondary process at around 120° C.

### Example 23

UHMWPE PM-200 was mixed with d-Limonene lubricant in 2:1 ratio (wt/vol) in a jar. The mixture was compressed in a preform press into a hollow cylindrical billet. The billet was loaded into a horizontal paste extruder and a 6.35 mm tubing die with a 3.2 mm mandrel was used for extruding large size tubing. The die had multiple heating zones, which were heated to set temperatures between 130° C. to 240° C. The extruded tubes were heat drawn 600% under tension at 120° C.

### Example 24

UHMWPE XM 221U was mixed with Isopar G lubricant in 1:1 ratio (wt/vol) in a jar. The mixture was added into the rheometer barrel and a 9.78 mm spider die with a 9.27 mm mandrel was used for extruding large size tubing. Only the die heating zone in the rheometer was turned on and set to a temperature of 180° C. The extruded tubes were heat drawn about 700% under tension in a secondary process at around 120° C.

### Example 25

UHMWPE XM 221U was mixed with d-Limonene lubricant in 3:1 ratio (wt/vol) in a jar. The mixture was compressed in a preform press into a hollow cylindrical billet. The billet was loaded into a horizontal paste extruder and a 6.35 mm tubing die with a 6.22 mm mandrel was used for extruding tubing. The die had multiple heating zones, which were heated to set temperatures between 60° C. to 220° C. The tubes were heat drawn about 600% under tension in a secondary process at around 120° C.

### Example 2

UHMWPE XM 221U was mixed with d-Limonene lubricant in 3:1 ratio (wt/vol) in a jar. The mixture was compressed in a preform press into a hollow cylindrical billet. The billet was loaded into a horizontal paste extruder and a 6.35 mm tubing die with a 6.22 mm mandrel was used for extruding tubing. The die had multiple heating zones, which were heated to set temperatures between 60° C. to 220° C. The tubes were heat drawn in-line during extrusion at around 120° C.

### Example 27

UHMWPE XM 221U extruded tubes from Example 24, without being subjected to in-line drawing, were heat drawn about 400% under tension in a secondary process at around 120° C.

### Example 28

UHMWPE XM 221U and GUR 211 resins were mixed in a 1:2 ratio (wt/wt) in a jar and d- Limonene was added as a lubricant to the resin mixture in 2:1 ratio (wt/vol). The mixture was compressed in a preform press into a hollow cylindrical billet. The billet was loaded into a horizontal paste extruder and a 6.35 mm tubing die with a 6.09 mm mandrel was used for extruding tubing. The die had multiple heating zones, which were heated to set temperatures between 60° C. to 240° C. The extruded tubes were heat drawn about 300% under tension in a secondary process at around 120° C.

### Example 29

UHMWPE XM 221U and GUR 211 resins were mixed in a 2:1 ratio (wt/wt) in a jar and Isopar G was added as a lubricant to the resin mixture in 3:1 ratio (wt/vol). The mixture was compressed in a preform press into a hollow cylindrical billet. The billet was loaded into a horizontal paste extruder and a 6.35 mm tubing die with a 6.09 mm mandrel was used for extruding tubing. The die had multiple heating zones, which were heated to set temperatures between 60° C. to 240° C. The extruded tubes were heat drawn about 300% under tension in a secondary process at around 120° C.

### Example 30

UHMWPE PM-200 was mixed with d-Limonene lubricant in 2:1 ratio (wt/vol) in a jar. The mixture was compressed in a preform press into a hollow cylindrical billet. The billet was loaded into a horizontal paste extruder and a 6.35 mm tubing die with a 6.09 mm was used for extruding tubing. The die had multiple heating zones, which were heated to set temperatures between 60° C. to 240° C. The extruded tubes were heat drawn 500% under tension in a secondary process at around 120° C.

### Example 31

UHMWPE PM-200 was mixed with d-Limonene lubricant in 3:1 ratio (wt/vol) in a jar. The mixture was compressed in a preform press into a hollow cylindrical billet. The billet was loaded into a horizontal paste extruder and a 6.35 mm tubing die with a 6.09 mm was used for extruding tubing. The die had multiple heating zones, which were heated to set temperatures between 60° C. to 260° C. The extruded tubes were heat drawn 400% under tension in a secondary process at around 120° C.

### Example 32

UHMWPE PM-200 was mixed with 5% (wt/wt) Epolene C-16P in a jar and d-Limonene was added as a lubricant to the resin mixture in 3:1 ratio (wt/vol). The mixture was compressed in a preform press into a hollow cylindrical billet. The billet was loaded into a horizontal paste extruder and a 6.35 mm tubing die with a 6.22 mm mandrel was used for extruding tubing. The die had multiple heating zones, which were heated to set temperatures between 30° C. to 180° C. The extruded tubes were heat drawn about 300% under tension in a secondary process at around 120° C.

### Example 33

UHMWPE PM 200 and irradiated UHMWPE PM 200 (10 MRad) resins were mixed in a 1:1 ratio (wt/wt) in a jar and 5% (wt/wt) Epolene C-16P was added to the UHMWPE resin mixture. d-Limonene was added as a lubricant to the resin mixture in 3:1 ratio (wt/vol). The mixture was compressed in a preform press into a hollow cylindrical billet. The billet was loaded into a horizontal paste extruder and a 6.35 mm tubing die with a 6.09 mm mandrel was used for extruding tubing. The die had multiple heating zones, which were heated to set temperatures between 30° C. to 180° C. The extruded tubes were heat drawn about 300% under tension in a secondary process at around 120° C.

### Example 34

UHMWPE XM 221U was mixed with 10% (wt/wt) powdered Orevac 18300M in a jar and d-Limonene was added as a lubricant to the resin mixture in 3:1 ratio (wt/vol). The mixture was compressed using a preform press into a hollow cylindrical billet. The billet was loaded into a horizontal paste extruder and a 6.35 mm tubing die with a 6.09 mm mandrel was used for extruding tubing. The die had multiple heating zones, which were heated to set temperatures between 30° C. to 180° C. The extruded tubes were heat drawn about 500% under tension in a secondary process at around 120° C.

### Example 35

UHMWPE PM 200 and irradiated UHMWPE PM 200 (10 MRad) resins were mixed in a 4:1ratio (wt/wt) in a jar and 5% (wt/wt) Epolene C-16P was added to the UHMWPE resin mixture. d-Limonene was added as a lubricant to the resin mixture in 3:1 ratio (wt/vol). The mixture was compressed in a preform press into a hollow cylindrical billet. The billet was loaded into a horizontal paste extruder and a 6.35 mm tubing die with a 6.22 mm mandrel was used for extruding tubing. The die had multiple heating zones, which were heated to set temperatures between 30° C. to 180° C. The tubes were subjected to testing as extruded without any additional draw-down process.

TABLE 1
List of Resins Used

| Resin | Grade |
|---|---|
| UHMWPE | Mipelon ™ PM-200 |
| UHMWPE | Mipelon ™ XM-221U |
| UHMWPE | Celanese GUR ® 4056-3 |
| UHMWPE | Celanese GUR ® 2122 |
| UHMWPE | Celanese GUR ® 4022-6 |
| PTFE | Polyflon F-201 |
| EVA | Microthene FE53200 |
| MA-LLDPE | OREVAC ® 18300M |
| PE Wax | Epolene ® C-16P |

TABLE 2
Mechanical Properties of UHMWPE Ribbon and Monofilament Samples

| | | | Tensile Properties | | | Storage | | | |
|---|---|---|---|---|---|---|---|---|---|
| | Dimensions | | Tensile | Stress @ | Strain @ | Modulus | | | COF |
| | Width | Thickness | Modulus | Break | Break | 23° C. | 40° C. | | in |
| | (mm) | (mm) | (MPa) | (MPa) | (%) | (MPa) | (MPa) | COF | Saline |
| Ex. 1 | 7.17 | 0.36 | 1320 | 65 | 12 | 1270 | 1080 | 0.15 | — |
| Ex. 2 | 6.01 | 0.38 | 1275 | 75 | 75 | 1300 | 1090 | 0.11 | — |
| Ex. 3 | 6.97 | 0.39 | 1090 | 50 | 13 | 1150 | 985 | 0.12 | — |
| Ex. 4 | 5.44 | 0.31 | 2365 | 180 | 47 | 1580 | 1370 | 0.11 | — |
| Ex. 5 | 6.00 | 0.34 | 2600 | 215 | 61 | 2300 | 1910 | 0.15 | — |
| Ex. 6 | 4.99 | 0.32 | 1750 | 175 | 112 | 1500 | 1280 | 0.17 | — |
| Ex. 7 | 5.84 | 0.33 | 2360 | 210 | 59 | 3250 | 2670 | 0.14 | 0.05 |
| Ex. 8 | 6.30 | 0.38 | 1560 | 140 | 56 | 1250 | 1040 | 0.14 | 0.04 |
| Ex. 9 | 7.54 | 0.47 | 2530 | 200 | 61 | 1650 | 1340 | 0.09 | — |
| Ex. 10 | 7.19 | 0.42 | 1845 | 145 | 30 | 1425 | 1200 | 0.16 | — |
| Ex. 11 | 6.73 | 0.39 | 2200 | 165 | 93 | 2500 | 2210 | 0.14 | — |
| Ex. 12 | 8.91 | 2.26 | 400 | 60 | 87 | — | — | — | — |
| Ex. 13 | 6.18 | 2.17 | 300 | 70 | 65 | — | — | — | — |
| Ex. 14 | 9.32 | 2.16 | 350 | 70 | 71 | — | — | — | — |
| Ex. 15 | 7.29 | 1.84 | 600 | 100 | 43 | — | — | — | — |
| Ex. 16 | 14.3 | 0.10 | 6630 | 200 | 4 | — | — | 0.14 | 0.05 |
| Ex. 17 | 0.91 | — | — | — | — | 2510 | 2000 | — | — |
| Ex. 18 | 0.43 | — | 4530 | 330 | 11 | 6200 | 5140 | — | — |
| Ex. 19 | 0.78 | — | 2300 | 200 | 19 | 2850 | 2300 | — | — |
| Ex. 20 | 2.95 | — | — | — | — | — | — | — | — |
| Ex. 21 | 3.68 | — | 500 | 80 | 48 | — | — | — | — |

TABLE 3
Mechanical Properties of UHMWPE Tubing Samples

| | | | | | | | | Flexural | | |
|---|---|---|---|---|---|---|---|---|---|---|
| | Dimensions | | Tensile Properties | | | Storage | | Stress @ | | |
| | | Wall | Tensile | Stress @ | Strain @ | Modulus | | 0.5% | | COF |
| | OD | Thickness | Modulus | Break | Break | 23° C. | 40° C. | Strain | | in |
| | (mm) | (mm) | (MPa) | (MPa) | (%) | (MPa) | (MPa) | (MPa) | COF | Saline |
| Ex. 22 | 3.02 | 0.46 | 1600 | 150 | 42 | 1190 | 970 | — | — | — |
| Ex. 23 | 3.42 | 0.09 | — | — | — | — | — | — | — | — |
| Ex. 24 | 1.94 | 0.049 | 1150 | 100 | 28 | 1270 | 950 | 0.22 | — | — |
| Ex. 25 | 1.96 | 0.047 | 3000 | 290 | 12 | 1540 | 1180 | 0.10 | 0.16 | — |
| Ex. 26 | 2.82 | 0.051 | 1150 | 190 | 30 | 1450 | 1100 | — | — | — |
| Ex. 27 | 2.09 | 0.043 | 1850 | 150 | 15 | 1725 | 1380 | 4.24 | — | — |
| Ex. 28 | 2.42 | 0.085 | 1325 | 90 | 20 | 1240 | 990 | — | — | — |
| Ex. 29 | 2.73 | 0.075 | 1130 | 110 | 26 | 790 | 630 | 0.06 | 0.13 | — |
| Ex. 30 | 1.95 | 0.04 | 2280 | 340 | 17 | 1195 | 925 | 0.89 | — | — |
| Ex. 31 | 2.87 | 0.05 | 1620 | 340 | 23 | 1350 | 1010 | 0.04 | 0.11 | — |
| Ex. 32 | 4.12 | 0.03 | 1060 | 140 | 32 | 770 | 625 | — | 0.18 | — |
| Ex. 33 | 2.70 | 0.07 | 1870 | 150 | 26 | 1260 | 1000 | — | — | — |
| Ex. 34 | 2.36 | 0.04 | 1220 | 125 | 17 | — | — | — | 0.15 | 0.06 |
| Ex. 35 | 4.87 | 0.07 | 1680 | 85 | 6 | 650 | 480 | — | 0.15 | 0.06 |

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. An Ultra High Molecular Weight Poly (ethylene) (UHMWPE) tube comprising:
a. an average wall thickness of 0.2 mm or less; and
b. a tensile stress at break greater than 40 MPa; and
c. a storage modulus of greater than 500 MPa at 23° C.

2. The UHMWPE tube of claim 1, wherein the UHMWPE tube is prepared via extrusion of a billet comprising lubricant and UHMWPE resin through an annular die.

3. The UHMWPE tube of claim 2, wherein the lubricant is selected from the group consisting of d-limonene, naphtha, Isopar G, Isopar M, or any combination thereof.

4. The UHMWPE tube of claim 1, wherein the UHMWPE tube is prepared via extrusion over a metallic or non-metallic wire or mandrel.

5. The UHMWPE tube of claim 4, wherein the metallic or non-metallic wire or mandrel and UHMWPE tube are both substantially cylindrical in shape.

6. The UHMWPE tube of claim 1, wherein the average wall thickness is 0.1 mm or less.

7. The UHMWPE tube of claim 1, wherein the average wall thickness of the tube is 0.005 mm to 0.1 mm.

8. The UHMWPE tube of claim 1, wherein the tube exhibits a change in the storage modulus between 23° C. and 40° C. of 70 MPa/° C. or less.

9. The UHMWPE tube of claim 1, comprising an inner surface with a coefficient of friction against stainless steel of less than 0.2.

10. The UHMWPE tube of claim 9, wherein a difference in the coefficient of friction between 23° C. and 40° C. is ≤0.1.

11. The UHMWPE tube of claim 1, comprising an inner surface with a coefficient of friction against stainless steel in saline of less than 0.1.

12. The UHMWPE tube of claim 11, wherein a difference in the coefficient of friction in saline between 23° C. and 40° C. is ≤0.1.

13. The UHMWPE tube of claim 1, consisting essentially of UHMWPE.

14. The UHMWPE tube of claim 1, comprising UHMWPE and a particulate filler, wherein the particulate filler is present in a concentration of less than 50% by weight, based on a weight of the UHMWPE tube.

15. The UHMWPE tube of claim 1, comprising UHMWPE and a particulate filler, wherein the particulate filler is present in a concentration of less than 20% by weight, based on a weight of the UHMWPE tube.

16. The UHMWPE tube of claim 14, wherein the particulate filler is a filler to impart radiopacity, strength, or hydrophilicity.

17. The UHMWPE tube of claim 1, further comprising one or more additives selected from the group consisting of antioxidants, antimicrobials, processing aids, sip aids, and colorants.

18. The UHMWPE tube of claim 1, wherein the UHMWPE tube comprises one or more additional polymeric materials other than UHMWPE, wherein the one or more additional polymer materials are present in a concentration of less than 50% by weight, based on a weight of the UHMWPE tube.

19. The UHMWPE tube of claim 1, wherein the UHMWPE tube comprises one or more additional polymeric materials other than UHMWPE, wherein the one or more additional polymer materials are present in a concentration of less than 20% by weight, based on a weight of the UHMWPE tube.

20. The UHMWPE tube of claim 18, wherein the one or more additional polymer materials are selected from modified polyethylene and ethylene vinyl acetate tie resins.
